(19) United States
(12) Patent Application Publication
(10) Pub. No.: US 2025/0281749 A1
(43) Pub. Date: Sep. 11, 2025

(54) NEURAL INTERFACE TECHNOLOGY FOR COMMUNICATING WITH CLINICALLY UNRESPONSIVE PATIENTS

(71) Applicant: PRECISION NEUROSCIENCE CORPORATION, New York, NY (US)

(72) Inventors: Craig MERMEL, New York, NY (US); Benjamin I. RAPOPORT, New York, NY (US); Elton HO, New York, NY (US)

(73) Assignee: PRECISION NEUROSCIENCE CORPORATION, New York, NY (US)

(21) Appl. No.: 19/075,143

(22) Filed: Mar. 10, 2025

Related U.S. Application Data
(60) Provisional application No. 63/562,998, filed on Mar. 8, 2024.

(51) Int. Cl. A61N 1/36

(52) U.S. Cl. CPC A61N 1/36067; A61N 1/36135

(57) ABSTRACT

A method and system for temporarily restoring communication ability in clinically unresponsive patients with cognitive motor dissociation. The system can include decoding recorded brain signals in response to commands to determine a communication state of the patient.

### PRIORITY

The present application claims priority to U.S. Provisional Patent Application No. 63/562,998, titled NEURAL INTERFACE TECHNOLOGY FOR COMMUNICATING WITH CLINICALLY UNRESPONSIVE PATIENTS, filed Mar. 8, 2024, which is hereby incorporated by reference herein in its entirety.

### BACKGROUND

Brain-computer interfaces have shown promise as systems for restoring, replacing, and augmenting lost or impaired neurological function in a variety of contexts, including paralysis from stroke and spinal cord injury, blindness, and some forms of cognitive impairment. Multiple innovations over the past several decades have contributed to the potential of these neural interfaces, including advances in the areas of applied neuroscience and multichannel electrophysiology, mathematical and computational approaches to neural decoding, power-efficient custom electronics and the development of application-specific integrated circuits, as well as materials science and device packaging. Nevertheless, the practical impact of such systems remains limited, with only a small number of patients worldwide having received highly customized interfaces through clinical trials.

High bandwidth brain-computer interfaces are being developed to enable the bidirectional communication between the nervous system and external computer systems in order to assist, augment, or replace neurological function lost to disease or injury. A brain-computer interface should be able to accurately decode electrophysiologic signals recorded from individual neurons, or populations of neurons, and correlate such activity with one or more sensory stimuli or intended motor response. For example, such a system can record activity from the primary motor cortex in an animal or a paralyzed human patient and attempt to predict the actual or intended movement in a specific body part; or the system can record activity from the visual cortex and attempt to predict both the location and nature of the stimuli present in the patient's visual field.

Furthermore, brain-penetrating microelectrode arrays have facilitated high-spatial-resolution recordings for brain-computer interfaces, but at the cost of invasiveness and tissue damage that scales with the number of implanted electrodes. In some applications, softer electrodes have been used in brain-penetrating microelectrode arrays; however, it is not yet clear whether such approaches offer a substantially different tradeoff as compared to conventional brain-penetrating electrodes. For this reason, non-penetrating cortical surface microelectrodes represent a potentially attractive alternative and form the basis of the system described here. In practice, electrocorticography (ECOG) has already facilitated capture of high quality signals for effective use in brain-computer interfaces in several applications, including motor and speech neural prostheses. Higher-spatial-resolution micro-electrocorticography (u ECoG) therefore represents a promising combination of minimal invasiveness and improved signal quality.

Recent studies have demonstrated that approximately 15-25% of clinically unresponsive patients are not in fact comatose and can hear and comprehend verbal communication, which can be referred to as cognitive motor dissociation (CMD). CMD describes a subset of patients who exhibit volitional brain activity as detected via neuroimaging (e.g., functional magnetic resonance imaging (fMRI) or electroencephalography (EEG)), but no detectable command-following behaviors. CMD refers to a subset of patients who appear unresponsive (showing no purposeful behavior) yet exhibit volitional brain activity in response to commands, as detected by functional MRI or EEG. In essence, it is a state of ‘covert consciousness’ where cognitive processes are present despite the loss of any motor output. Patients suffering from CMD cannot verbally respond or carry out actions due to disruption of the neural pathways downstream of the cortical regions responsible for motor planning and execution. Aside from the general undesirability of allowing patients who can hear and comprehend verbal commands to remain in a state in which they cannot communicate with other individuals, not being able to communicate with these patients can lead to choosing treatment plans that are not best suited to their needs. Further, studies have demonstrated that clinically unresponsive patients with CMD have better ultimate outcomes than comatose patients. Therefore, being able to determine which patients have CMD and which are truly comatose can help in selecting proper treatment plans for the different types of clinically unresponsive patients. Conventional neural interfaces could be used to assist in the diagnosis of CMD; however, conventional neural interfaces are highly invasive and cause damage to the cortical surface due to their penetrating electrodes, which makes them undesirable to use for diagnostic purposes. Therefore, minimally invasive neural interfaces would be beneficial because they could be used in these instances where conventional neural interfaces are impractical.

Conventional techniques have not been sufficient for diagnosing CMD or restoring communication with patients suffering from CMD. In particular, bedside behavioral assessments, fMRI, and/or EEG are the conventional diagnostic tools for diagnosing CMD. Standardized bedside neurobehavioral exams, such as the Coma Recovery Scale-Revised (CRS-R), have been used to detect any subtle purposeful behaviors. However, even with careful exams, some conscious patients may show no overt responses. Misdiagnosis is common if assessments are not rigorous. Further, guidelines emphasize treating reversible confounders and performing serial standardized exams to improve accuracy. Therefore, conventional bedside behavioral assessments have been inadequate.

Task-based fMRI has also been used to detect covert command-following in the brain. For example, patients may be instructed to imagine specific movements (e.g., “imagine opening and closing your hand”) and then fMRI has been used to attempt to reveal characteristic activation in motor-planning areas if the patients are following commands covertly. Such paradigms have shown that some patients with no outward responses can willfully modulate their brain activity in predictable ways. In one study, 25% of unresponsive patients produced sustained, relevant fMRI (or EEG) responses to commanded tasks. These results confirm earlier case reports that a small but significant subset of vegetative patients can demonstrate awareness through brain imaging. Similarly, EEG-based paradigms at the bedside can also pick up volitional brain responses to commands. For instance, asking the patient to imagine moving their hand while recording EEG may reveal specific changes (such as event-related potentials or shifts in sensorimotor rhythms) indicating they attempted the task. EEG is more accessible than fMRI and can be implemented at the ICU bedside repeatedly. However, fMRI and EEG are unlikely to be able to decode patient intention with sufficient accuracy to restore communication with patients that are clinically unresponsive, but not comatose.

Accurate diagnosis of CMD is critically important for patient care and outcomes. Identifying that a patient has preserved cognition despite an inability to move or speak can drastically change management. It helps avoid the premature withdrawal of life-sustaining treatment in patients who have a realistic chance of improvement. Further, knowledge of consciousness can guide discussions with families about goals of care, ensuring that decisions (such as continuing intensive therapy verse comfort measures) are well-informed. There are also rehabilitation implications: patients with CMD tend to have better long-term outcomes than those without any signs of consciousness. For example, one study found that among initially unresponsive brain-injured patients, those diagnosed with CMD were significantly more likely to achieve functional recovery within 12 months. In that cohort, CMD status was an independent predictor of a shorter time to recovery and higher functional ability, and it helped identify patients who could benefit from rehabilitation efforts. In summary, recognizing CMD allows clinicians to provide more precise prognostic counseling and to pursue therapeutic avenues for patients who might otherwise be presumed hopeless. Ethically, it acknowledges the patient's internal awareness and affords them an opportunity (through specialized means) to communicate and participate in their care to the extent possible.

### SUMMARY

The present disclosure is directed to systems and methods for communicating with a patient who is clinically unresponsive.

In some embodiments, there is provided a method for communicating with a patient that is clinically unresponsive via a cortical interface device, the method comprising: implanting the cortical interface device at a region of a motor cortex of a patient; guiding the patient through a training exercise while recording brain signals via the cortical interface device; initiating decoding of the recorded brain signals; instructing the patient to imagine performing a motor function; and communicating with the patient based on the results of the decoded brain signals.

In some embodiments, there is provided a method for communicating with a patient that is clinically unresponsive, the method comprising: guiding the patient through a training exercise while recording brain signals via a cortical interface device at a region of a motor cortex of the patient; initiating decoding of the recorded brain signals; instructing the patient to imagine performing a motor function; and assessing a communication state of the patient based on the results of the decoded brain signals.

In some embodiments, the method further comprises determining, based on the recorded brain signals, that the motor cortex of the patient is responding to the training exercise.

In some embodiments, the cortical interface device is a first cortical interface device and the region of the motor cortex is a first region of the motor cortex, the method further comprising placing a second cortical interface device at a second region of the motor cortex of the patient.

In some embodiments, the method further comprises removing the cortical interface device within thirty days of placement.

In some embodiments, the motor function comprises squeezing a hand or throwing an object.

In some embodiments, the motor function is associated with a binary response.

In some embodiments, there is provided a computer-implemented method for communicating with a patient that is clinically unresponsive using a cortical interface device surgically implanted adjacent to a motor cortex at a cortical surface of the patient, the cortical interface comprising an electrode array for recording neural signals from the cortical surface, the method comprising: receiving, by a computer system, brain signals from the cortical interface device, wherein the brain signals correspond to an action being imagined by the patient, wherein the action corresponds to the motor cortex; training, by the computer system, a machine learning model on the received brain signals to decode the action; determining, by the computer system, whether the trained machine learning model exhibits at least a threshold performance in decoding the action; and in response to the trained machine learning model exhibiting at least the threshold performance, indicating, by the computer system, via the user interface, whether the patient is imagining the action. In some embodiments, the training of the machine learning model comprises transfer learning.

In some embodiments, there is provided a system for communicating with a patient that is clinically unresponsive, the system comprising: a cortical interface device to be surgically implanted adjacent to a motor cortex at a cortical surface of the patient, the cortical interface comprising an electrode array for recording neural signals from the cortical surface; and a computer system communicably coupled to the cortical interface, the computer system comprising a user interface, a processor, and a memory, the memory storing instructions that, when executed by the processor, cause the computer system to: receive brain signals from the cortical interface device, wherein the brain signals correspond to an action being imagined by the patient, wherein the action corresponds to the motor cortex; train a machine learning model on the received brain signals to decode the action; determine whether the trained machine learning model exhibits at least a threshold performance in decoding the action; and in response to the trained machine learning model exhibiting at least the threshold performance, indicate, via the user interface, whether the patient is imagining the action. In some embodiments, the machine learning model comprises a convolutional neural network.

### FIGURES

FIG. 1 depicts a block diagram of a secure neural device data transfer system, in accordance with an embodiment of the present disclosure.

FIG. 2 depicts a diagram of a neural device, in accordance with an embodiment of the present disclosure.

FIG. 3 depicts a flow diagram of a process for communicating with a patient that is clinically unresponsive, in accordance with an embodiment of the present disclosure.

FIG. 4 depicts a flow diagram of a computer-implemented process for communicating with a patient that is clinically unresponsive, in accordance with an embodiment of the present disclosure.

FIG. 5 depicts graphs demonstrating binary classification performance based on intraoperative data, in accordance with an embodiment of the present disclosure.

### DETAILED DESCRIPTION

The present disclosure is generally directed to surgical systems and methods for temporarily restoring communication ability in clinically unresponsive patients. In particular, the present disclosure is directed to using a temporarily implanted neural device to communicate using brain signals.

Disclosed herein is a system of restoring communication with clinically unresponsive patients using a minimally invasive neural device that can be temporarily placed within a patient. Embodiments can include the method of restoring communication with a clinically unresponsive patient. Embodiments can also include the system which collects and decodes the brain signals in response to the method of restoring communication. In some embodiments, the system can utilize a Layer 7 Cortical Interface™ to communicate with the clinically unresponsive patient.

### Neural Device Systems

Conventional neural devices typically include electrode arrays that penetrate a subject's brain in order to sense and/or stimulate the brain. However, the present disclosure is directed to the use of non-penetrating neural devices, i.e., neural devices having electrode arrays that do not penetrate the cortical surface. Such non-penetrating neural devices are minimally invasive and minimize the amount of impact on the subject's cortical tissue. This allows for implementing the neural device for clinically unresponsive patients as it can be trained and used shortly after being implanted on the brain. Neural devices can sense and record brain activity, receive instructions for stimulating the subject's brain, and otherwise interact with a subject's brain as generally described herein.

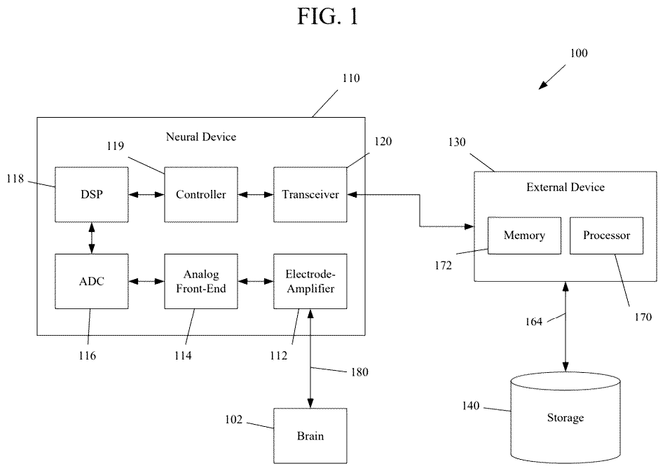

Referring now to FIG. 1, there is shown a diagram of an illustrative system 100 including a neural device 110 that is communicatively coupled to an external device 130. The external device 130 can include any device to which the neural device 110 can be communicatively coupled, such as a computer system or mobile device (e.g., a tablet, a smartphone, a laptop, a desktop, a secure server, a smartwatch, a head-mounted virtual reality device, a head-mounted augmented reality device, or a smart inductive charger device). The external device 130 can include a processor 170 and a memory 172. In some embodiments, the external device 130 can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure).

In some embodiments, the electrode array 180 of the neural device 110 can have electrodes that are sufficiently small and spaced at sufficiently small distances in order to define a high-density electrode array 180 that can, accordingly, capture high resolution electrocortical data. Such high-resolution data can be used to resolve electrographic features that can otherwise not be identified using lower resolution electrode arrays. In some embodiments, the electrodes of the electrode array 180 can be from about 10 μm to about 500 μm in width. In one illustrative embodiment, the electrodes of the electrode array 180 can be about 50 μm in width. In some embodiments, the electrodes of the electrode array 180 can be spaced by about 200 μm (i.e., 0.2 mm) to about 3,000 μm (i.e., 3 mm). In illustrative one embodiment, adjacent electrodes of the electrode array 180 can be spaced by about 400 μm.

The neural device 110 can include a range of electrical or electronic components. In the illustrated embodiment, the neural device 110 includes an electrode-amplifier stage 112, an analog front-end stage 114, an analog-to-digital converter (ADC) stage 116, a digital signal processing (DSP) stage 118, and a transceiver stage 120 that are communicatively coupled together. The electrode-amplifier stage 112 can include an electrode array, such as is described below, that is able to physically interface with the brain 102 of the subject in order to sense brain signals and/or apply electrical signals thereto. The analog front-end stage 114 can be configured, amplify signals that are sensed from or applied to the brain 102, perform conditioning of the sensed or applied analog signals, perform analog filtering, and so on. The front-end stage 114 can include, for example, one or more application-specific integrated circuits (ASICs) or other electronics. The ADC stage 116 can be configured to convert received analog signals to digital signals. The DSP stage 118 can be configured to perform various DSP techniques, including multiplexing of digital signals received via the electrode-amplifier stage 112 and/or from the external device 130. For example, the DSP stage 118 can be configured to convert instructions from the external device 130 to a corresponding digital signal. The transceiver stage 120 can be configured to transfer data from the neural device 110 to the external device 130 located outside of the body of the subject 102.

In some embodiments, the neural device 110 can further include a controller 119 that is configured to perform various functions, including compressing electrophysiologic data generated by the electrode array 180. In various embodiments, the controller 119 can include hardware, software, firmware, or various combinations thereof that are operable to execute the functions described below. In one embodiment, the controller 119 can include a processor (e.g., a microprocessor) executing instructions stored in a memory. In another embodiment, the controller 119 can include a field-programmable gate array (FPGA) or application-specific integrated circuit (ASIC).

In various embodiments, the stages of the neural device 110 can provide unidirectional or bidirectional communications (as indicated in FIG. 1) by and between the neural device 110 and the external device 130. In various embodiments, one or more of the stages can operate in a serial or parallel manner with other stages of the system 100. It can further be noted that the depicted architecture for the system 100 is simply intended for illustrative purposes and that the system 100 can be arranged differently (i.e., components or stages can be connected in different manners) or include additional components or stages.

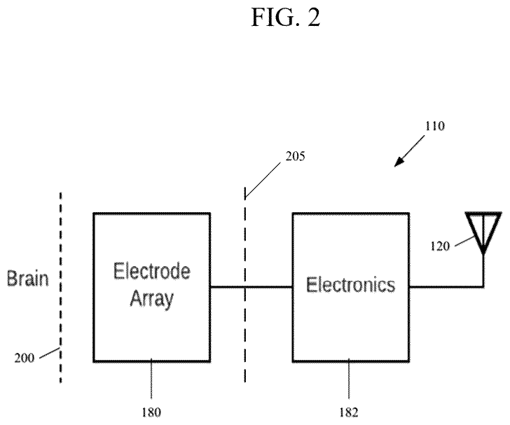

Referring now to FIG. 2, the electrode array 180 can include non-penetrating cortical surface microelectrodes (i.e., the electrode array 180 does not penetrate the brain 200). Accordingly, the neural device 110 can provide a high spatialresolution, with minimal invasiveness and improved signal quality. The minimal invasiveness of the electrode array 180 is beneficial because it allows the neural device 110 to be used with larger population of patients than conventional brain implants, thereby expanding the application of the neural device 110 and allowing more individuals to benefit from brain-computer interface technologies. Furthermore, the surgical procedures for implanting the neural devices 110 are minimally invasive, reversible, and avoid damaging neural tissue. In some embodiments, the electrode array 180 can be a high-density microelectrode array that provides smaller features and improved spatial resolution relative to conventional neural implants.

In some embodiments, the neural device 110 includes an electrode array configured to stimulate or record from neural tissue adjacent to the electrode array, and an integrated circuit in electrical communication with the electrode array, the integrated circuit having an analog-to-digital converter (ADC) producing digitized electrical signal output. In some embodiments, the ADC or other electronic components of the neural device 110 can include an encryption module, such as is described below. The neural device 110 can also include a wireless transmitter (e.g., the transceiver 120) communicatively coupled to the integrated circuit or the encryption module and an external device 130. The neural device 110 can also include, for example, control logic for operating the integrated circuit or electrode array 180, memory for storing recordings from the electrode array, and a power management unit for providing power to the integrated circuit or electrode array 180.

Decoding Neural Signals for Communicating with Clinically Nonresponsive Patients

The present disclosure relates to systems and methods for communicating with patients who are clinically unresponsive. In certain implementations, the brain-computer interface system described herein can enable communication with patients who have CMD. CMD refers to a condition where patients retain cognitive function but are unable to produce voluntary motor responses due to neurological injury. These patients can appear to be in a vegetative or minimally conscious state, but brain imaging studies have shown that some can understand and respond to commands mentally despite being unable to do so physically.

Brain-computer interfaces (BCIs) can be used as a form of digital-bypass therapy that is aimed to restore function to individuals with different forms of neurologic injury or disability. Because the BCIs that have been explored to date are invasive and tissue-damaging, the vast majority of the focus in the literature and commercial efforts has been towards creating permanently implantable BCIs with an aim towards helping those with permanent neurologic injury, for example, patients with cervical spinal cord injury, brainstem stroke, or end-stage ALS. However, hundreds of thousands of patients in the United States end up in a temporary state of clinical unconsciousness arising from various conditions, including traumatic brain injury, stroke, infection, or drug overdose. One challenge in the management of these patients is their inability to communicate with the care team or their families, making it challenging to monitor and predict their neurologic recovery and/or attend to their needs. Indeed, recent studies have demonstrated that approximately 15-25% of clinically unresponsive patients can hear and comprehend verbal commands but cannot carry out those commands due to temporary disruption of the motor pathways downstream of the cortical regions responsible for motor planning and execution. Such temporary states of CMD are highly similar to those that are treated in more permanent neurologic injury; however, little attention has been paid to how to leverage intact cortical function for improved clinical communication in CU patients.

Because certain clinically unresponsive patients are not in fact comatose, it would be beneficial to be able to differentiate between such patients and those patients who are truly comatose. However, there are currently no existing medical techniques for being able to reliably identify clinically unresponsive, but non-comatose patients (i.e., patients with CMD). To remedy these problems, the neural interfaces described herein can be used for this purpose because they can be implanted in a minimally invasive manner and can be used on a temporary basis because the electrodes do not penetrate the cortical surface and, therefore, do not cause any damage to the brain. One technique for using the neural interfaces described herein to identify non-comatose, clinically unresponsive patients would be to (i) implant the neural interfaces on the cortical surface at a location corresponding to a particular motor cortex associated with an action, (ii) instruct the patient to imagine performing the action (since the patient would definitionally not be able to perform the action since they are clinically unresponsive), and (iii) train a machine learning model to determine whether the patient is in fact imagining performing the action, which would indicate that the patient is able to hear and understand verbal commands and, thus, is not comatose despite being clinically unresponsive.

Being able to communicate with patients experiencing CMD, even slightly, can decrease the challenges of monitoring and predicting the patient's neurological recovery. Without being able to determine the brain activity of the patient, a clinically unresponsive patient would seem the same as a patient in a coma and, thus, would be treated in an identical manner. Studies have shown that clinically nonresponsive patients generally have better ultimate outcomes than patients in a coma. Being able to detect and determine the patient's ability to understand verbal communication through the systems and processes described herein can help to determine which patients are clinically nonresponsive verse those who are truly in a coma. This determination can assist doctors to make the right treatment decisions for each patient.

Another benefit of the systems and processes described herein is the ability to do short-term implantations due to the non-invasive implantation and the improvements in neural decoding. Previously, neural decoding algorithms required many days of training data, which incentivized long-term implantations over more short-term use cases. With these methods and systems, clinically relevant communication function can be established within the first day of implantation.

The systems and methods described herein can allow for detecting and decoding neural signals associated with imagined speech or motor actions in CMD patients. This can enable these patients to communicate by imagining speaking words or performing motor tasks, even though they cannot physically speak or move. In some cases, the system can include a cortical interface device, such as the neural device 110 described above, that can be temporarily implanted on the surface of a patient's brain to record neural activity. The system can also include computer hardware and software for processing and decoding the recorded brain signals to determine the patient's intended communication. By potentially restoring some ability to communicate, this technology can improve diagnosis, care, and quality of life for patients with disorders of consciousness who retain covert awareness. The minimally invasive and temporary nature of the system can allow it to be used as both a diagnostic tool and a communication aid.

In embodiments for communicating with clinically nonresponsive patients, the cortical interface device can comprise a flexible, ultra-thin polymer substrate embedded with high-density microelectrodes designed to conform to the cortical surface. In some cases, the polymer substrate can be made of a biocompatible material such as polyimide or parylene. The microelectrodes can be fabricated from conductive materials like gold, platinum, or iridium oxide. In some implementations, the cortical interface device can placed at various regions of the motor cortex, including the motor cortex regions corresponding to upper extremities, lower extremities, and facial muscles. The cortical interface device can also be positioned on premotor cortical regions in some implementations. The high-density microelectrode array can allow for recording of neural activity with high spatial and temporal resolution. In some cases, the microelectrodes can have a diameter ranging from 10 to 100 micrometers and can be spaced 100 to 500 micrometers apart. This configuration can enable detection of signals from individual neurons or small populations of neurons. The flexibility and ultra-thin nature of the substrate can allow the cortical interface device to conform closely to the curved surface of the brain. This conformability can help maintain consistent contact between the electrodes and cortical tissue, potentially improving signal quality and stability. In some implementations, the cortical interface device can incorporate additional features such as integrated flexible electronics for signal amplification and multiplexing. This can help reduce the number of wired connections required and minimize tethering forces on the brain.

The cortical interface device can be designed for temporary implantation, with a duration ranging from several days to a few weeks. As noted above, the cortical interface devices described herein use nonpenetrating electrodes and, accordingly, the device can be removable without causing damage to the underlying cortical tissue, allowing for its use as both a diagnostic tool and short-term communication aid. The ability for temporary, removeable applications of the cortical interface device, without causing any damage to the underlying cortical tissue, is a significant improvement over cortical interface technologies that make use of penetrating (i.e., depth) electrodes because penetrating electrodes inherently cause damage to the cortical tissue, which limits their ability to be used in temporary applications.

The neural signal processing unit can be configured to extract volitional intent signals from recorded cortical activity and differentiate these signals from background neural noise. In some cases, the neural signal processing unit can include components for signal amplification, filtering, feature extraction, and decoding. The signal amplification stage can boost the weak electrical signals recorded by the cortical interface device. In some cases, low-noise amplifiers with high input impedance can be used to minimize signal distortion. Signal filtering can be applied to remove artifacts and isolate frequency bands of interest. In some cases, bandpass filters can be used to focus on specific frequency ranges associated with motor imagery or speech intent, such as mu (8-12 Hz) and beta (13-30 Hz) rhythms.

Feature extraction techniques can be employed to identify relevant characteristics of the neural signals. In some cases, time-domain features like signal amplitude and spectral power in specific frequency bands can be extracted. Other approaches can include wavelet decomposition or common spatial pattern analysis. The decoding stage can utilize machine learning algorithms to translate the extracted features into intended actions or speech. In some cases, linear classifiers, support vector machines, or artificial neural networks can be used for this purpose. The decoding process can be time-synchronized to external cues presented to the patient, allowing for precise temporal alignment between neural activity and intended actions.

In some implementations, adaptive algorithms can be employed to account for non-stationarities in neural signals and improve decoding performance over time. These algorithms can continuously update the decoding model based on new data, potentially enhancing the system's ability to interpret the patient's intentions accurately. The neural signal processing unit can also incorporate techniques for real-time signal processing and decoding. In some cases, parallel processing architectures or field-programmable gate arrays (FPGAs) can be used to achieve low-latency performance, enabling responsive communication with the patient.

To differentiate volitional intent signals from background neural noise, various noise reduction techniques can be applied. In some cases, independent component analysis (ICA) or principal component analysis (PCA) can be used to separate signal sources and isolate task-related neural activity. The neural signal processing unit can also implement methods for handling signal artifacts caused by movement, electrical interference, or physiological processes. In some cases, adaptive filtering techniques or artifact rejection algorithms can be employed to maintain signal quality and decoding accuracy.

The brain-computer interface computer system (e.g., external device 130) can be configured to translate processed neural signals into various communication outputs. This module can serve as the interface between the neural signal processing unit and external devices or user interfaces that allow the patient to communicate. In some cases, the brain-computer interface computer system can generate text-based outputs. For example, the computer system can translate decoded neural signals into individual letters, words, or complete sentences. These text outputs can be displayed on a screen visible to caregivers and family members, allowing them to read the patient's intended communication. The brain-computer interface computer system can also produce auditory outputs in some implementations. Decoded neural signals can be converted into synthesized speech, allowing the patient to communicate verbally through a speaker system. This can enable more natural conversation-like interactions between the patient and others. In some cases, the computer system can generate visual outputs beyond text. For example, decoded signals can be used to control an on-screen cursor or select icons representing basic needs or emotions. This can allow patients to navigate simple graphical user interfaces to express themselves.

The brain-computer interface computer system can incorporate adaptive algorithms to improve communication accuracy over time. These algorithms can learn from the patient's neural patterns and refine the translation process to better interpret the patient's intentions. In some implementations, the computer system can implement error correction mechanisms. For example, if the system detects low confidence in a decoded output, it can prompt the patient for confirmation or clarification before displaying the result.

As described above, the cortical interface device can be designed for minimally invasive placement and positioning along the cortical interface device, reducing risks associated with traditional craniotomy-based implantation. The system can also include tools and techniques for removing the cortical interface device at the end of the monitoring period. These can be designed to allow for safe extraction of the electrode array without causing damage to the underlying cortical tissue. In some cases, the placement system can incorporate neuronavigation technology to guide the positioning of the electrode array. This can involve registering preoperative medical imaging scans with the patient's anatomy in the operating room to ensure accurate targeting of specific cortical regions.

The minimally invasive placement system can be designed with modularity to accommodate different sizes and configurations of electrode arrays. This flexibility can allow for customization based on individual patient anatomy and the specific cortical regions to be monitored.

The system can incorporate a machine learning algorithm designed to refine the detection and interpretation of neural activity over time, potentially improving communication accuracy and reliability. This algorithm can be implemented as part of the neural signal processing unit or the brain-computer interface module. In some cases, the machine learning algorithm can utilize supervised learning techniques. During initial training sessions, the patient can be asked to imagine specific actions or words while their neural activity is recorded. These labeled data sets can then be used to train the algorithm to recognize patterns associated with different imagined actions or speech. The algorithm can employ various machine learning models, such as support vector machines, random forests, or artificial neural networks. In some implementations, deep learning architectures like convolutional neural networks or recurrent neural networks can be used to capture complex spatiotemporal patterns in the neural data. As the system is used over time, the machine learning algorithm can continuously update its models based on new data. This adaptive learning process can allow the system to account for changes in the patient's neural patterns, which can occur due to factors such as neuroplasticity or fluctuations in the patient's condition. In some cases, the algorithm can incorporate transfer learning techniques. This approach can allow the system to leverage knowledge gained from other patients or healthy individuals to improve performance, even with limited data from the current patient. The machine learning algorithm can also implement techniques for handling non-stationarities in the neural signals. For example, it can use adaptive filters or sliding window approaches to track changes in the signal statistics over time. In some implementations, the algorithm can employ ensemble methods, combining predictions from multiple models to improve overall accuracy and robustness. This approach can help mitigate the impact of individual model errors or biases.

The system can include mechanisms for evaluating the performance of the machine learning algorithm. In some cases, a portion of the recorded data can be set aside as a validation set to assess the algorithm's accuracy. The system can use metrics such as classification accuracy, F1 score, or area under the receiver operating characteristic curve to quantify performance. Based on these performance evaluations, the system can automatically adjust hyperparameters or model architectures to optimize performance. In some cases, this process can involve techniques such as Bayesian optimization or genetic algorithms to efficiently search the parameter space. The machine learning algorithm can also incorporate techniques for handling class imbalance, which can occur if certain imagined actions or words are used more frequently than others. Methods such as oversampling, undersampling, or synthetic data generation can be employed to address this issue. In some implementations, the algorithm can use active learning techniques. The system can identify instances where it has low confidence in its predictions and prompt the patient or caregiver for additional labeled examples, focusing the learning process on the most informative data points. The machine learning algorithm can also implement techniques for feature selection and dimensionality reduction. This can help identify the most relevant aspects of the neural signals for decoding, potentially improving computational efficiency and reducing overfitting. In some cases, the algorithm can incorporate unsupervised learning techniques to discover latent structures in the neural data. Methods such as clustering or dimensionality reduction can be used to identify patterns that are not apparent in the labeled training data. As the machine learning algorithm refines its interpretation of neural activity, it can enable more complex communication tasks over time. For example, the system can initially focus on simple binary choices and gradually progress to word or sentence-level decoding as accuracy improves. In some cases, the algorithm can incorporate multi-task learning, simultaneously optimizing for multiple objectives such as accuracy, speed, and patient effort. This approach can help balance different aspects of communication performance. To improve transparency and interpretability, the system can include visualization tools that allow clinicians to inspect the decision-making process of the machine learning algorithm. This can help build trust in the system and provide insights into the patient's neural patterns. In some cases, the machine learning algorithm can incorporate federated learning techniques, allowing the system to benefit from data across multiple patients while maintaining privacy and data security. This approach can be particularly useful for improving performance in rare conditions or with limited data.

The system can be configured in different ways to accommodate various clinical needs and use cases. In some cases, the system can be implemented as a temporary percutaneous (wired) device. This configuration can involve the cortical interface device being implanted on the brain surface with wires extending through the scalp to connect to external processing hardware. The temporary percutaneous configuration can be suitable for short-term use, such as when assessing a patient's candidacy for a more permanent implant or during acute care situations. In some cases, this setup can allow for easier removal or adjustment of the device if needed. The wired connection can provide high-bandwidth data transmission and reliable power supply to the implanted components. In other cases, the system can be configured as a permanent wireless implant for long-term use. This configuration can involve fully implanting all components, including the cortical interface device and a sealed unit containing processing hardware and a wireless transmitter. The wireless implant can communicate with external devices using radio frequency or other wireless protocols. The permanent wireless configuration can be appropriate for patients who require ongoing communication assistance and have demonstrated good outcomes with the temporary system. In some cases, this setup can offer greater mobility and convenience for the patient, as there are no percutaneous wires that could become damaged or infected. Each configuration can have specific advantages depending on the clinical context. For example, the temporary percutaneous system can be preferred in intensive care settings where frequent monitoring and adjustment are necessary. In contrast, the permanent wireless implant can be more suitable for patients in long-term care facilities or home environments where a lower-maintenance solution is desired. In some cases, a patient can transition from the temporary percutaneous configuration to the permanent wireless implant. This approach can allow for an initial trial period to assess the effectiveness of the brain-computer interface before committing to a more invasive permanent implantation. The choice between configurations can also depend on factors such as the patient's overall health status, the expected duration of use, and the availability of surgical resources for implantation and maintenance. In some cases, regulatory considerations can influence the selection of a particular configuration, as temporary and permanent implants can have different approval pathways. Both configurations can support similar functionality in terms of neural signal processing and communication interfaces. However, the specific implementation details, such as power management strategies and data transmission protocols, can differ between the wired and wireless versions of the system.

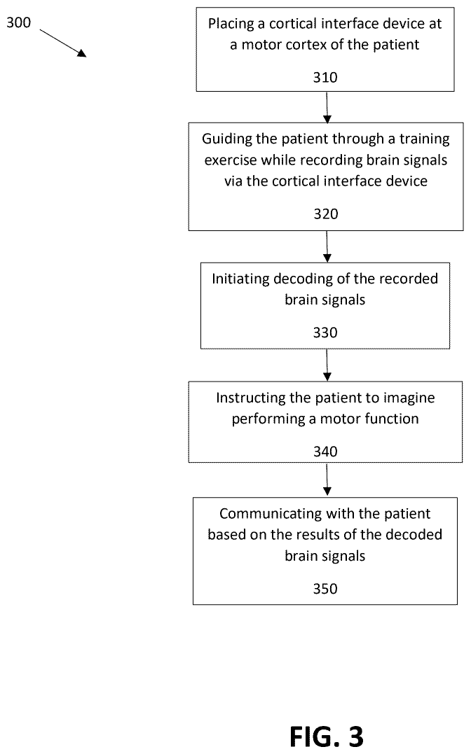

Referring to FIG. 3, there is shown a flow diagram of a process 300 for communicating with a patient that is clinically unresponsive, in accordance with an embodiment of the present disclosure. First, the patient can be medically stabilized and determined to be non-responsive by traditional clinical methods. In some embodiments, the process can include step 310. In step 310, a cortical interface device can be placed at a region of a motor cortex of the patient. The region of the motor cortex can correspond to a certain body part. In some embodiments, the cortical interface device can be the neural device 110 as described above in reference to FIGS. 1-2. In some embodiments, the cortical interface device can be placed at any number of the motor regions of the cortex of the patient's brain. In some embodiments, there can be at least two cortical interface devices adjacent at least two respective regions of the motor cortex of the patient's brain. In some embodiments, one cortical interface device is placed adjacent the patient's dominant hand region of the motor cortex and another cortical interface device is placed adjacent the patient's speech/language region of the motor cortex. In some embodiments, the cortical interface device can be implanted using minimally invasive surgical techniques, such as are described in U.S. patent application Ser. No. 18/434,008, titled MINIMALLY INVASIVE INSERTION SYSTEM FOR NEURAL INTERFACES, filed Feb. 6, 2024, which is hereby incorporated by reference herein in its entirety. In some embodiments, the devices can be wired out transcutaneously to external electronics or a temporary wireless implant can be placed, depending on the expected duration of the implant and other clinical considerations.

In step 320, the patient can be guided through a training exercise while recording brain signals via the cortical interface device. In some embodiments, there can be a first phase in which the goal can be to establish that the patient's cortex is responding to verbal commands to either move or speak, to determine whether adequate BCI communication can be established. In some embodiments, the first phase can be used to determine, based on the recorded brain signals, that the motor cortex is responding to the training exercise. In some embodiments, if adequate BCI communication cannot be established, the cortical interface device can be left in place as a prognostic tool and the same protocol can be repeated over several days to determine if the patient recovers sufficient function to attempt BCI communication. In some embodiments, if adequate BCI communication can be established, then additional training data can be collected. This additional training data can include instructing the patient to attempt to speak specific words or perform certain motor actions at a prompt.

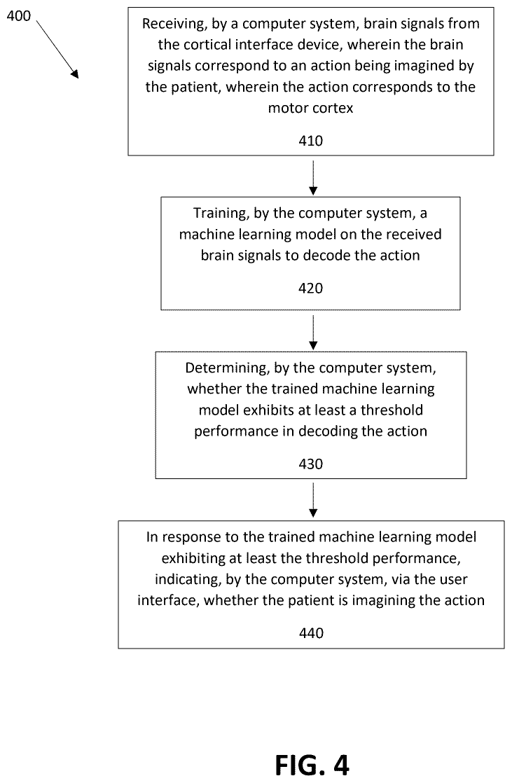

In step 330, decoding of the recorded brain signals can be initiated. In some embodiments, the process for decoding the recorded brain signals can include the process 400 shown in FIG. 4 and described below. In some embodiments, once sufficient training data has been collected, as in step 320, a decoding algorithm can be initiated, which can be trained on the patient's data. In some embodiments, the decoding algorithm can require at most several hours of training data due to the efficient training methods of transfer learning described below in process 400. A validation dataset can also be collected with which to compare the decoding algorithm's results. In some embodiments, the validation dataset can be collected by instructing the patient to repeat a subset of the training commands and a ground truth can be established. The cortical interface device can record brain signals in response to the commands. Decoding of these recorded brain signals can be initiated. Once the recorded brain signals have been decoded, the results of the decoding can be compared to the ground truth. If the accuracy is greater than a pre-specified performance threshold, the algorithm can be said to be trained and can be used. In some embodiments, the pre-specified performance threshold can be greater than 50%. In some embodiments, the pre-specified performance threshold can be about 75%. In some embodiments, the pre-specified performance threshold can be about 90%. If the accuracy is less than the pre-specified performance threshold, additional training data can be collected and step 330 can be repeated until sufficient performance is attained, or the cortical interface device is explanted.

In step 340, the patient can be instructed to imagine performing a motor function. This instruction can be any combination of imagining speaking and/or imagining moving a body part, such as wiggling toes, moving hands, etc. In some embodiments, the instruction can be a command, a question, or any type of reasonable communication to a patient. A person of ordinary skill will understand the types of reasonable communications one can have with a patient.

In step 350, communication can be established with the patient based on the results of the decoded brain signals. In some embodiments, the decoding algorithm can be used to communicate with the patient in a guided fashion, such as in a question-and-answer format. If the decoding algorithm determines that the patient responded to a prompt and determines that the accuracy is greater than a pre-specified performance threshold, then the decoding algorithm can display the results and/or a communicated response on a user interface. In some embodiments, the user interface can be on a local workstation adjacent the patient. In some embodiments, the user interface can provide a real-time read-out of the patient's communication state. In some embodiments, the patient can also be able to proactively request assistance. In some embodiments, the patient can be instructed to imagine a certain motor function to get assistance. For example, the patient can be instructed to imagine wiggling their toes if they need assistance. If the patient then imagines wiggling their toes, the results of the decoded brain signals can show that the communication state of the patient can be one of needing assistance.

As the device is used for communication as disclosed, the device can be monitored to ensure the device remains in active calibration as the brain changes. In some embodiments, daily performance checks can be performed to ensure the decoding remains accurate. In some embodiments, if the performance drops below the pre-specified performance threshold, additional rounds of training can be required to get the decoding algorithm back in calibration.

In some embodiments, the device can be explanted once thirty days has passed.

Referring now to FIG. 4, there is shown a flow diagram of a computer-implemented process 400 for training a machine learning model to facilitate communication with a patient that is clinically unresponsive, in accordance with an embodiment of the present disclosure. As described above, the neural interfaces described herein can be used to facilitate communication with clinically unresponsive patients. Further, the systems described herein can implement machine learning models and/or techniques in order to decode patient brain signals to assess patients' communication states (i.e., whether they are comatose) and, in certain cases, identify patients' responses to verbal commands and/or questions. As one aspect of these systems and methods, the machine learning models can be calibrated to patients so that they can be used to decode patient brain signals. Accordingly, the computer systems described herein can be communicably coupled to the cortical interface device placed adjacent a region of the motor cortex of the brain, as described above in connection with the process 300 shown in FIG. 3. In some embodiments, the computer system can include a user interface, a processor, and a memory. In some embodiments, the memory can store instructions that, when executed by the processor, cause the computer system to perform the computer-implemented process 400. Once the cortical interface device is in place, the process 400 can be performed.

As described above, the patient can be guided through a training exercise whereby verbal commands and/or questions are provided to the user to assess whether the clinically unresponsive patients are able hear and comprehend the verbal communications. Accordingly, in step 410, a computer system can receive brain signals from the cortical interface device in association with verbal questions being posed to the patient and/or verbal commands being given to the patient. In some embodiments, the brain signals correspond to an action being imagined by the patient. In some embodiments, the imagined action corresponds to the region of the motor cortex at which the cortical interface device is placed. In some embodiments, the action can include imagining speaking or moving any part of the patient's body, such as imagining moving a limb and/or imagining moving a digit.

In step 420, the computer system can train a machine learning model on the received brain signals to decode the action. In some embodiments, the computer system can receive the commands given to the patient to imagine a certain action in order to align the action to the signals. In some embodiments, the machine learning model can include a convolutional neural network. In some embodiments, the training can include transfer learning. In some embodiments, the machine learning model can require at most several hours of training data and can be trained within one day.

In step 430, the computer system can determine whether the trained machine learning model exhibits at least a threshold performance in decoding the action. In some embodiments, the threshold performance level can be greater than 50% accuracy. In some embodiments, the threshold performance level can be about 75% accuracy. In some embodiments, the threshold performance level can be about 90% accuracy. In some embodiments, the threshold performance level can be different for different uses or different decoding tasks. For example, if the task for which the machine learning model is being used is simply determining whether the patient can understand commands, the threshold performance level can be lower. Conversely, if the task for which the machine learning model is being used is asking the patient to consent to perform particular treatments or to select different treatment options, a high threshold performance level can be used. In some embodiments, if the trained machine learning model is determined to exhibit at least the threshold performance level, then it can subsequently be used to decode the patient's brain signals to determine whether the patient is imagining the action(s) that the machine learning model has been trained to decode.

In step 440, the computer system can indicate (e.g., via a user interface) whether the brain signals correspond to the action that is associated with the particular brain region at which the neural interface has been placed (e.g., whether the patient is imagining the action or otherwise attempting to perform the action) using the trained and validated machine learning model. In some embodiments, the machine learning model can be trained to provide a binary-based indication. For example, if the patient is instructed to imagine speaking if he or she needs more medication, a determination that the patient imagined speaking by the decoding machine learning model can create an indication of, “yes” on the user interface. Otherwise, the machine leaning model can output a “no.” In some embodiments, the indication can be a response based on the question or command and the response given. For example, if the patient is instructed to imagine moving his or her right hand if more pain medication is desired or imagine moving his or her left hand if more pain medication is not desired, a determination that the patient imagined moving their right hand can create an indication of, “I would like more pain medication,” or a similar statement, on the user interface. In some embodiments, the computer system can be trained with certain commands, such that the patient can proactively request assistance without responding to a question or command. For example, the patient can be told that imagining wiggling their toes will call the nurse. Then, if the patient subsequently imagines wiggling their toes, the computer system can receive the brain signals corresponding to the imagined action via the neural interface, make a determination based on the trained machine learning model whether the received brain signals correspond to the trained action, and indicate on the user interface that the patient is requesting a nurse.

Additional information regarding decoding techniques described herein can be found in International Application No. PCT/US2024/017647, titled DATA COMPRESSION FOR NEURAL SYSTEMS, filed Feb. 28, 2024; U.S. International Patent Application No. PCT/US23/35623, titled SELF-CALIBRATING NEURAL DECODING, filed Oct. 20, 2023; and U.S. International Patent Application No. PCT/US23/77626, titled DATA-EFFICIENT TRANSFER LEARNING FOR NEURAL DECODING APPLICATIONS, filed Oct. 24, 2023, which are hereby incorporated by reference herein in their entireties.

### Illustrative Clinical Protocol

In order to elucidate the systems and techniques described herein, an illustrative clinical protocol is outlined below. It should be noted that this is only one potential clinical implementation and the systems and techniques described herein could be implemented, either in the clinic or in practice, in different ways.

First, likely CMD candidates can be identified. Patients can initially be evaluated with standard CMD diagnostic methods before implant consideration. This can include repeated thorough bedside examinations (using tools like the CRS-R) and, when available, ancillary tests such as EEG-based command-following paradigms or fMRI mental imagery tasks. Patients who remain behaviorally unresponsive despite optimal assessment, especially those who show any suggestive evidence of consciousness on those tests, can be considered for a neural implant. For example, a patient who cannot follow any commands outwardly, but nonetheless demonstrates brain activation in response to verbal commands (on EEG or fMRI) would be a strong candidate. In practice, roughly one-quarter of clinically unresponsive patients may show signs of covert consciousness when rigorously tested with combined neuroimaging and electrophysiological approaches. Those who demonstrate such covert responses (indicating possible CMD), but cannot reliably communicate can be the primary target group for a trial of the neural implant. If a patient has no hint of awareness on any modality (and especially if the cause of injury carries a poor prognosis), the risks of an invasive device may outweigh the benefits.

Next, medical stability and timing can be analyzed. Eligible patients should be medically stable enough to undergo a minor neurosurgical procedure. This means any confounding factors for unresponsiveness have been addressed and corrected as much as possible (e.g. treating acute metabolic disturbances, discontinuing sedating medications, ensuring adequate oxygenation and perfusion). Because the techniques described herein are based on detecting neural responses to verbal commands, optimizing arousal in patients should be a key goal. Accordingly, sedative drips should be weaned off and the patient allowed to cycle through wake-sleep to the extent possible. Typically, screening for an implant would take place after the initial acute phase of brain injury, once intracranial pressure is controlled and life-threatening issues are managed. Many studies have included patients in the ICU within days of injury for advanced assessments, so an implant could be considered relatively early (within the first 1-2 weeks) if the patient remains unresponsive but medically stable. Early introduction of the device (with use for up to 30 days) is ideal in the ICU because this is the period when critical decisions are often made. The ability to detect consciousness or communicate during this window can impact treatment plans (e.g., decisions about surgery, tracheostomy, or continuation of intensive support) rather than discovering awareness only later when some options may no longer be available.

Next, inclusion and exclusion considerations can be analyzed. Not every disorder-of-consciousness patient will be a candidate for even a minimally invasive BCI, such as described herein. Ideal candidates would be those in a vegetative state (also termed unresponsive wakefulness syndrome) or in a minimally conscious state without reliable communication, with evidence or strong suspicion of CMD. Patients with traumatic brain injury (TBI) are often the focus of these efforts, as studies suggest they more frequently exhibit covert consciousness and have a higher likelihood of recovery compared to patients with diffuse anoxic injury. On the other hand, patients whose condition is due to widespread anoxic brain damage or those who show signs of irreversible neurological devastation (e.g. absent brainstem reflexes or isoelectric EEG) would not be candidates. Each case should be evaluated for contraindications to surgery, such as uncontrolled coagulopathy or severe systemic instability. If the patient meets clinical criteria, the care team (including neurologists, neurosurgeons, and the patient's surrogates) must also consider the ethical aspects; in particular, there should be a clear intent and consent to pursue all measures to establish communication/diagnosis. Surrogate decision-makers must agree that a temporary implant aligns with the patient's values and the goals of care. In summary, screening for implant eligibility involves integrating current CMD diagnostic findings with medical readiness. The patient most suitable for a reversible implant is one who is likely conscious but locked-in, medically stable for a procedure, and for whom gaining communication or diagnostic certainty would substantially influence care.

Candidates that have satisfied the screening criteria can then be fitted with a BCI, as described herein. Once implanted, the cortical interface can immediately start recording the patient's neural activity at a very high resolution. Compared to scalp EEG, the signal obtained is far clearer (due to no skull attenuation) and more spatially detailed, capturing localized cortical dynamics in real time. To utilize the implant for diagnosing CMD, clinicians would engage the patient in the same mental tasks used in EEG/fMRI paradigms, but now observe the brain's responses through the high-density ECoG grid. For example, the care team might periodically instruct the patient to think about squeezing their right hand or visualize throwing a ball. The BCI system (e.g., having 1,024 contact points) can detect subtle voltage changes across the motor and premotor cortex as the patient attempts these tasks. If the patient is indeed conscious and following the command internally, the interface would show a distinguishable pattern of activation (such as a change in the power of specific brainwave frequencies or a distinct spatial activation signature) corresponding to the instructed activity. Early studies using this technology in neurosurgical patients have demonstrated the ability to map and display cortical activity with sub-millimeter resolution. Thus, even a very focal activation (which may be be undetectable on standard ICU EEG) could be picked up by the BCI array described herein. The presence of a task-specific brain response would confirm the diagnosis of CMD, serving as direct evidence that the patient's cortex is processing commands and trying to respond. In essence, the implant acts as a highly sensitive diagnostic monitor for consciousness, reducing false negatives that could occur with less sensitive tools.

Beyond diagnosing covert awareness, the implanted interface provides a means to facilitate communication with a patient who cannot speak or move. Over the course of the up to 30-day implant period, the medical team can calibrate a brain-computer communication system tailored to the individual. This typically involves selecting a reliable neural signal that the patient can modulate intentionally. For instance, one strategy is to have the patient focus on a mental task (e.g., motor imagery of the hand or tongue) to signify “yes” and do nothing (or focus on a different mental image) to signify “no.” The BCI device would feed the neural data into a computer running machine-learning algorithms to distinguish these two states. Because the electrode array is high-channel-count and sits directly on the cortex, it can capture even subtle differentials in brain activity when the patient is attempting one mental task versus another. With training and feedback, many patients can learn to produce a consistent brain signal for a binary response. Prior research has shown that even patients in a completely locked-in state (no eye or any movement at all) due to ALS were able to communicate “yes” and “no” answers using an implanted BCI that decoded their cortical signals. In an ICU setting, a patient with CMD implanted with the neural interface described herein could conceivably answer simple questions in real time, such as indicating “yes” or “no” about being in pain, understanding their situation, or consenting to a procedure. Additionally, if the patient demonstrates a robust control signal, it might be possible to set up an assistive communication interface (such as a speller board or tablet) where the patient's brain signals move a cursor or select letters. Given the short time frame and the acuity of illness, initial goals would likely focus on basic communication (i.e., yes/no or simple choices) to improve patient care. Even this limited communication channel can be tremendously meaningful: it provides the patient a way to express their needs and feelings, and it reassures family members that their loved one is “still there” and capable of interaction.

The use of the neural interface and techniques described herein can provide several benefits in an ICU setting for CMD patients. For example, the neural interface and techniques described herein can provide enhanced monitoring. Aside from communication, the high-resolution cortical recording can serve as an advanced monitoring tool. Brain-injured patients in the ICU are at risk for secondary neurological events such as subclinical seizures or cortical spreading depolarizations. These events often have no outward signs but can worsen outcomes if not treated. The BCI array's dense coverage can detect abnormal electrical activities (e.g., seizure patterns or spreading waves) that might be missed by routine EEG. This provides a valuable early-warning system and a window of opportunity for intervention (e.g., administering anti-epileptic drugs if a seizure pattern is detected). In essence, the implant can function as a continuous EEG with much higher fidelity, improving patient safety.

Further, the neural interface can be used for dynamic assessment of neurologic function. ICU neurological exams are limited when a patient is paralyzed or comatose. The BCI offers a way to dynamically assess the patient's residual cortical function over time. Clinicians can periodically engage the patient in command-following via the interface and track any improvements or changes in their responses. A patient who initially shows no response might begin to exhibit consistent brain responses after some days, indicating emerging recovery. This kind of monitoring could help gauge the trajectory of neurological recovery even in a non-communicative patient.

As another example, the neural interface and techniques described herein can be used for informed decision-making and patient autonomy. Having a communication channel, however basic, profoundly impacts the clinical management of an ICU patient. The patient can express their wishes or at least participate in simple decisions (like “Do you want to continue with this treatment?”). From an ethical standpoint, this respects the patient's autonomy by giving a voice to someone who would otherwise be voiceless. It can also prevent misunderstandings. For example, if a patient is in pain or distress, they could indicate that, prompting appropriate relief measures. Families and care teams often struggle with the uncertainty in disorders of consciousness. Thus, a BCI that allows the patient to signal yes/no provides much-needed clarity and reassurance. It can strengthen the therapeutic alliance and ensure that care plans align with the patient's own preferences as much as possible.

Finally, the neural interface and techniques described herein for establishing communication with CMD patients can provide psychological and rehabilitation benefits. For the patient, the mere act of successfully communicating can be psychologically beneficial. Regaining the ability to answer questions or interact, even through a device, can reduce feelings of entrapment and isolation. It may also stimulate cognitive pathways by essentially “exercising” the brain through purposeful effort, which some hypothesize could aid recovery. Early engagement through BCI might facilitate earlier participation in rehabilitation once the patient transitions out of ICU. At minimum, it keeps the patient mentally active, which is valuable in an otherwise low-stimulation environment.

### Task Classification Performance Results

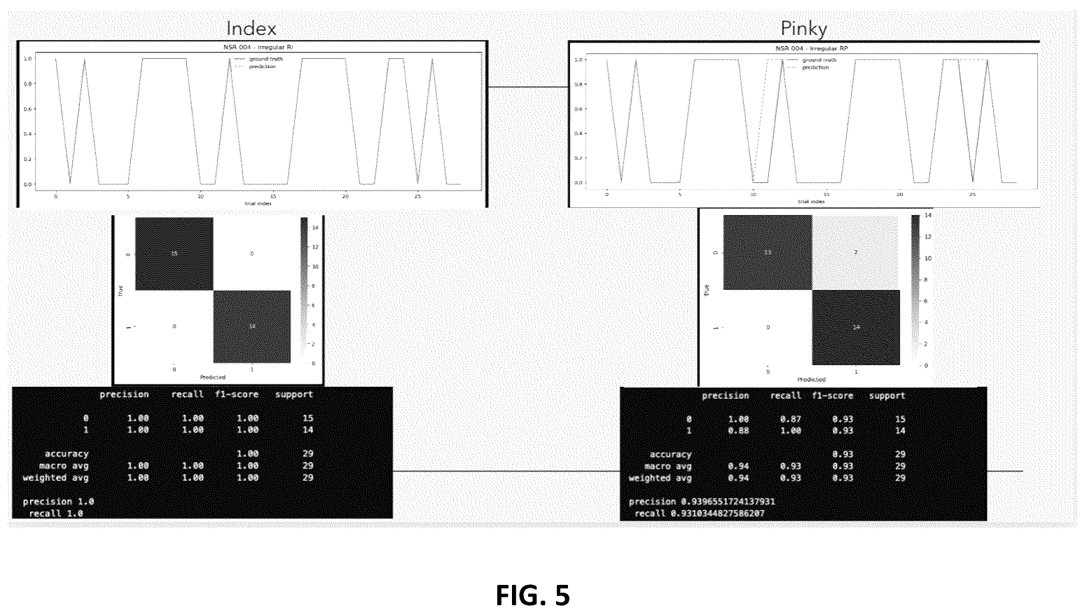

As described above, the systems and methods described herein can be used at least initially to facilitate binary (i.e., “yes” or “no”) communication with CMD patients by having them focus on imaging performing two different tasks, one of which is associated with one response (e.g., “yes”) and the other of which is associated with another response (e.g., “no”). Once calibrated to the patient, the BCI system can then output textual or audio output corresponding to the patient's binary responses to queries, thereby facilitating communication between the patient and care givers or family members. The BCI system described herein has been experimentally to demonstrate much higher accuracy on decoding tasks from intraoperative data compared to conventional techniques that do not utilize the high resolution, high bandwidth neural interfaces described herein. For example, FIG. 5 illustrates binary classification performance of the neural decoding models implemented by the BCI system described herein for detecting flexion/extension for individual fingers (in this case, the index finger and the pinky finger). Individual finger flexion/extension detection is conceptually similar to the cognitive imagery task that is used in existing non-invasive CMD protocols. In particular, FIG. 5 shows that by using only approximately five minutes of intraoperative data collected via the neural interfaces described herein, the machine learning algorithms implemented by the BCI system was able to achieve accuracies of 93-100%, as compared to EEG which has accuracies for individual questions of approximately 50-60%. EEG may be statistically different from chance, but is hardly accurate enough to be reliably used as a communication channel. Accordingly, the BCI system described herein has been experimentally validated as being capable of performing extremely accurately in two-class (i.e., binary) classification tasks, which is precisely the type of classification tasks that would be used for establishing initial communication with CMD patients using the techniques described herein. Further, the BCI system described herein has also been experimentally validated as exhibiting highly accurate performance on three-class classification tasks, thus can also be used to establish higher order communication with CMD patients once initial communication has been established.

This disclosure is not limited to the particular systems, devices, and methods described, as these can vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the term “clinically unresponsive” means a state of unresponsiveness, which includes comatose and cognitive motor dissociation, in which the patient seems to not be able to respond appropriately to stimuli.

As used herein, the term “comatose” means a state of unresponsiveness in which the patient cannot be aroused to respond appropriately to stimuli even with vigorous stimulation and shows no brain activity attempting to respond to the stimuli.

As used herein, the term “cognitive motor dissociation” means a state of unresponsiveness in which the patient can hear and comprehend verbal commands but cannot carry out those commands due to disruption of the motor pathways downstream of the cortex and shows brain activity attempting to respond to the commands.

As used herein, the singular forms “a,” “an,” and “the” include plural references, unless the context clearly dictates otherwise. Thus, for example, reference to a “protein” is a reference to one or more proteins and equivalents thereof known to those skilled in the art, and so forth.

As used herein, the term “about” means plus or minus 10% of the numerical value of the number with which it is being used. Therefore, about 75% means in the range of 65% to 85%.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “subject” includes, but is not limited to, humans and non-human vertebrates such as wild, domestic, and farm animals.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method for communicating with a patient that is clinically unresponsive via a cortical interface device, the method comprising:
implanting the cortical interface device at a region of a motor cortex of a patient;
guiding the patient through a training exercise while recording brain signals via the cortical interface device;
initiating decoding of the recorded brain signals;
instructing the patient to imagine performing a motor function; and
communicating with the patient based on the results of the decoded brain signals.

2. The method of claim 1, further comprising:
determining, based on the recorded brain signals, that the motor cortex of the patient is responding to the training exercise.

3. The method of claim 1, wherein the cortical interface device is a first cortical interface device and the region of the motor cortex is a first region of the motor cortex, the method further comprising:
placing a second cortical interface device at a second region of the motor cortex of the patient.

4. The method of claim 1, further comprising:
removing the cortical interface device within thirty days of placement.

5. The method of claim 1, wherein the motor function comprises squeezing a hand or throwing an object.

6. The method of claim 1, wherein the motor function is associated with a binary response.

7. A computer-implemented method for communicating with a patient that is clinically unresponsive using a cortical interface device surgically implanted adjacent to a motor cortex at a cortical surface of the patient, the cortical interface comprising an electrode array for recording neural signals from the cortical surface, the method comprising:
receiving, by a computer system, brain signals from the cortical interface device, wherein the brain signals correspond to an action being imagined by the patient, wherein the action corresponds to the motor cortex;
training, by the computer system, a machine learning model on the received brain signals to decode the action;
determining, by the computer system, whether the trained machine learning model exhibits at least a threshold performance in decoding the action; and
in response to the trained machine learning model exhibiting at least the threshold performance, indicating, by the computer system, via the user interface, whether the patient is imagining the action.

8. The computer-implemented method of claim 7, wherein the training, by the computer system, the machine learning model on the received brain signals to decode the action comprises transfer learning.

9. The computer-implemented method of claim 7, wherein the cortical interface device is a first cortical interface device, the region of the motor cortex is a first region of the motor cortex, and the brain signals are first brain signals, the computer-implemented method further comprising:
receiving second brain signals from a second cortical interface device at a second region of the motor cortex of the patient.

10. The computer-implemented method of claim 7, wherein the action comprises squeezing a hand or throwing an object.

11. The computer-implemented method of claim 7, wherein the action is associated with a binary response.

12. A system for communicating with a patient that is clinically unresponsive, the system comprising:
a cortical interface device to be surgically implanted at a motor cortex at a cortical surface of the patient, the cortical interface comprising an electrode array for recording neural signals from the cortical surface; and
a computer system communicably coupled to the cortical interface, the computer system comprising a user interface, a processor, and a memory, the memory storing instructions that, when executed by the processor, cause the computer system to:
receive brain signals from the cortical interface device, wherein the brain signals correspond to an action being imagined by the patient, wherein the action corresponds to the motor cortex;
train a machine learning model on the received brain signals to decode the action;
determine whether the trained machine learning model exhibits at least a threshold performance in decoding the action; and
in response to the trained machine learning model exhibiting at least the threshold performance, indicate, via the user interface, whether the patient is imagining the action.

13. The system of claim 12, wherein the machine learning model comprises a convolutional neural network.

14. The system of claim 12, wherein the cortical interface device is a first cortical interface device implanted at a first region of the motor cortex, the system further comprising:
a second cortical interface device to be surgically implanted at a second region of the motor cortex of the patient.

15. The system of claim 12, wherein the action comprises squeezing a hand or throwing an object.

16. The method of claim 12, wherein the action is associated with a binary response.
